(19) United States
(12) Patent Application Publication
(10) Pub. No.: US 2016/0135674 A1
(43) Pub. Date: May 19, 2016

(54) A MEASUREMENT PROBE

(71) Applicant: WATERFORD INSTITUTE OF TECHNOLOGY, Waterford (IE)

(72) Inventors: Colin Reidy, Limerick (IE); Brendan Phelan, Waterford (IE); Eoghan O'Donoghue, Waterford (IE); Ramesh Raghavendra, Waterford (IE); Patrick Carton, Waterford (IE)

(21) Appl. No.: 14/903,088

(22) PCT Filed: Jun. 30, 2014
(86) PCT No.: PCT/EP2014/063845

§ 371 Date: Jan. 6, 2016

(30) Foreign Application Priority Data
Jul. 8, 2013 (GB) 1312214.8

(51) Int. Cl. A61B 1/317; A61B 1/005; A61B 1/07; A61B 1/04; A61B 1/00; A61B 1/06

(52) U.S. Cl. CPC A61B 1/317; A61B 1/00009; A61B 1/0684; A61B 1/07; A61B 1/04; A61B 1/005; A61B 1/00101; A61B 1/00103

(57) ABSTRACT

A probe for measuring features in inaccessible locations comprising an elongate body adapted to be inserted into said inaccessible location, a probe tip being defined at a distal end thereof, a light source for transmitting light from said probe tip to illuminate a surface adjacent the probe tip, an image sensor for generating an image of the surface adjacent the probe tip and image processing means programmed to carry out digital imaging correlation upon subsequent images generated by said optical sensor to determine the motion of the probe tip.

This invention relates to a probe for measuring features in inaccessible locations, and in particular to an arthroscopic probe for measuring the dimensions of cartilage lesions.

Articular cartilage is an avascular soft tissue that covers the articulating bony ends of joints. During joint motion, cartilage acts as a lubricating and shock absorbing mechanism in the articulating joints and protects the underlying bony structures by minimising peak contact force at the joints. Once damaged, articular cartilage has limited or no ability to heal due to a lack of vasculature and often degenerates, leading to osteoarthritis.

The size of a cartilage lesion or other surface defect is an important guideline for the treatment options available to a patient. Therefore the measurement of a cartilage lesion is a critical stage in the diagnosis and treatment of the patient.

Arthroscopic diagnosis of cartilage lesions has traditionally been very inaccurate. Traditional methods comprise the use of endoscopes to view a cartilage lesion with the use of landmarks, such as probe tips of known width, whereby the surgeon locates the landmark in or adjacent the lesion estimates the dimensions of the lesion by a visual comparison of the size of the lesion with the size of the probe tip. This method is highly subjective and thus prone to gross error.

According to the present invention there is provided a probe for measuring features in inaccessible locations comprising an elongate body adapted to be inserted into said inaccessible location, a probe tip being defined at a distal end thereof, a light source for transmitting light from said probe tip to illuminate a surface adjacent the probe tip, an image sensor for generating an image of the surface adjacent the probe tip and image processing means programmed to carry out digital imaging correlation upon subsequent images generated by said image sensor to determine the motion of the probe tip.

The light source may comprise at least one LED or at least one laser diode.

In one embodiment the light source and/or the image sensor may be provided within the body of the probe, light being transmitted from the light source to the probe tip and from the probe tip to the optical sensor via respective light guides, such as optical fibres.

In an alternative embodiment the light source and/or the image sensor may be provided at or adjacent said probe tip.

Preferably the tip of the probe is provided with a lens for receiving reflected light from said surface and transmitting the resulting image to the image sensor. The image processing means may programmed to determine when said image is in focus. An indicator may be provided for indicating that said image is in focus, as determined by the image processing means. The lens may be recessed into the tip of the probe such that the distance between the lens and an outermost lip of the tip of the probe is equal to the focal distance of the lens such that the image of the surface is in focus when the tip of the probe is in contact with the surface.

The image sensor may comprise an array of light sensors, such as photodiodes, each light sensor defining a pixel of the resulting image.

Preferably the light source is adapted to illuminate the surface adjacent the tip of the probe at an oblique angle when the tip of the probe extends normal to the surface.

Preferably the orientation of the probe tip with respect to the probe body can be adjusted by up to 90° to allow the probe to be inserted into said inaccessible location from one side in a direction substantially parallel to the surface. The probe tip may be formed from a flexible or malleable material to facilitate such adjustment.

In one embodiment the probe comprises an arthroscopic probe for measuring the dimensions of a lesion or other defect in articular cartilage. However, numerous other applications are envisaged and a probe in accordance with the present invention may be utilised wherever it is desired to take measurements of the dimensions of features located in inaccessible locations, such as inside engines, pipelines or similar enclosed and/or inaccessible spaces.

A measurement probe in accordance with an embodiment of the present invention will now be described, by way of example only, with reference to the accompanying drawings, in which:—

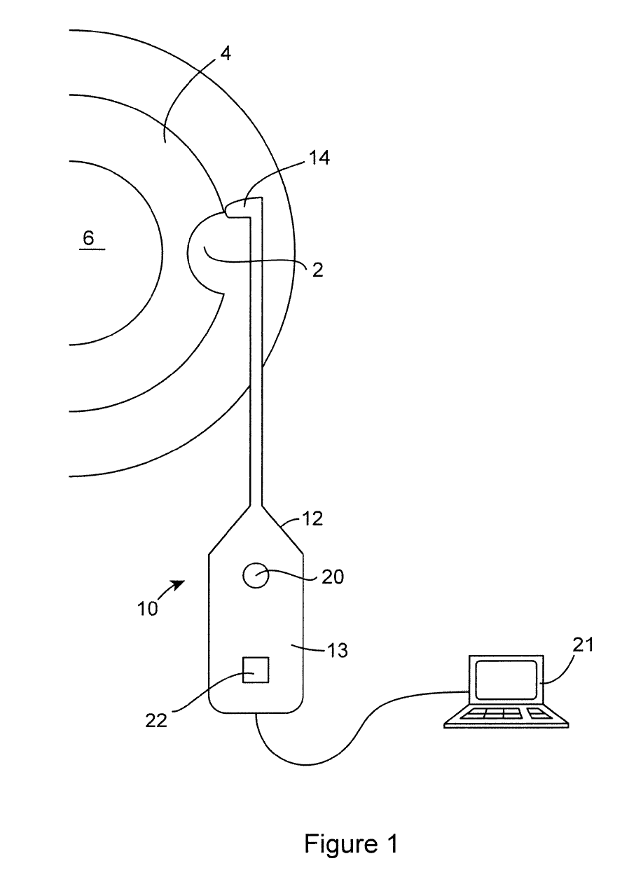

FIG. 1 is a schematic diagram of an arthroscopic probe in accordance with an embodiment of the present invention in use;

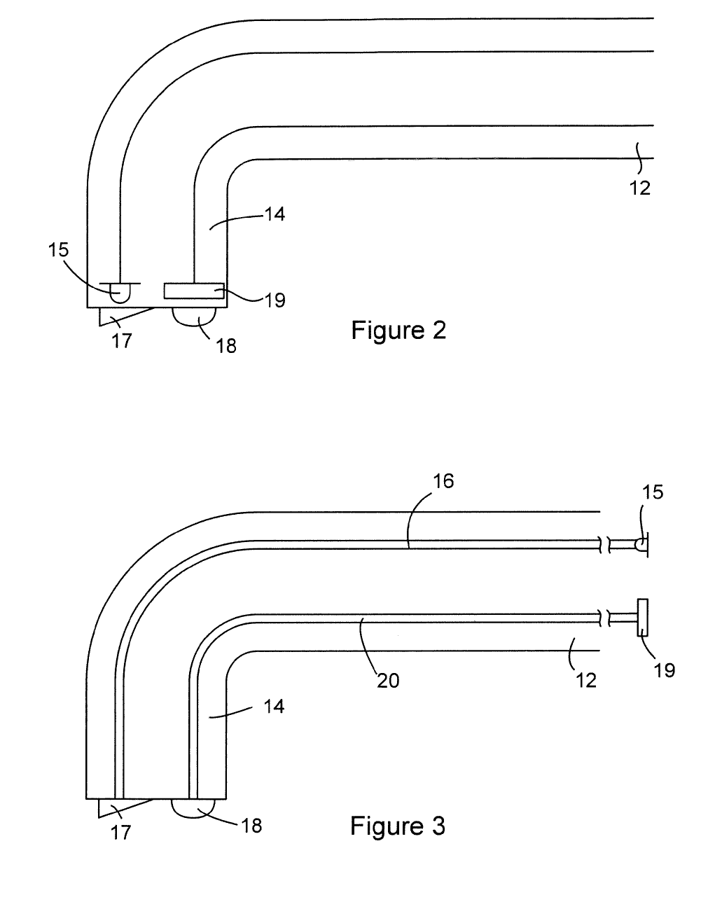

FIG. 2 is a sectional view through an end region of the probe of FIG. 1; and

FIG. 3 is a sectional view through an end region of a modified probe in accordance with a further embodiment of the invention.

An arthroscopic probe 10 in accordance with an embodiment of the present invention comprises an elongate body 12 having a handle 13 at a base end and a tip 14 at a distal end thereof. The tip 14 of the probe may extend substantially perpendicular to the body 12. In a preferred embodiment, the probe tip is formed from a flexible or malleable material permitting the orientation of the probe tip 14 with respect to the body 12 to be adjusted, as required. This may allow the probe to be inserted into an inaccessible location from one side in a direction substantially parallel to a surface to be viewed. A light source 15, such as one or more LEDs or a laser diode, is provided for illuminating a surface adjacent the tip 14 of the probe, preferably via a prism or window 17 mounted in the tip 14 of the probe 10, such that light is directed at a surface adjacent the tip of the probe at an oblique angle to said surface.

The light source 15 may be provided within the tip 14 of the probe 10, as shown in FIG. 2, or within the body 12 of the probe. In the latter case, as shown in FIG. 3, the light source 15 may communicate with the tip 14 of the probe via an optical fibre 16 extending through the hollow body 12 of the probe 10, the optical fibre 16 terminating at the prism or window 17 window mounted in the tip 14 of the probe for directing light onto a surface adjacent the probe.

Reflected light is collected by a lens 18 mounted in the tip 14 of the probe and focussed onto an image sensor 19. The image sensor 19 may be mounted in the tip 14 of the probe, adjacent the lens 18 (as shown in FIG. 2) or may be mounted within the body 12 of the probe, the image being transmitted to the image sensor 19 from the lens 18 via an optical fibre 20 (as shown in FIG. 3) extending through the body 12 of the probe 10.

The image sensor 19 may comprise an array of light sensors, for example photodiodes, each light sensor defining a pixel of the resulting image. An array of approximately 18 rows and 18 columns or light sensors may be provided such that the image

The image sensor 19 provides data to an image processing device, in the form of a micro-processor, which captures images a pre-determined intervals and performs digital image correlation on the captured images to determine the movement of the probe tip 14 over the surface and thus provide an accurate measurement of the distance moved by the tip 14 of the probe in the x and y directions. The movement of the tip 14 of the probe is determined in a similar manner to the movement tracking function of an optical mouse.

Preferably the image processing device is adapted to determine when the image received from the lens 18 is in focus (i.e. when the distance of the tip 14 of the probe from the adjacent surface is equal to the focal distance of the lens 18). An indicator 20, for example a light or LED, may be provided on the body 12 of the probe, preferably on the exterior of the handle 13, the image processing device or associated processing means being programmed to activate the indicator 20 when the image is in focus. Thus the indicator 20 can be used to track the edge of a feature to be measured, such a lesion in articular cartilage. The indicator 20 also serves to facilitate the positioning of the tip 14 of the probe at the correct distance from the surface to be measured.

Preferably the lens 18 is recessed into the tip 14 of the probe such that the distance between the lens 18 and an outer lip of the tip 14 is equal to the focal length of the lens, whereby the image of the surface will be in focus when the outer lip of the tip 14 is in contact with the surface.

The probe 10 may be connected to a computer 21, by a wired or wireless data connection. Such computer 21 may be programmed to interpret the data received from the probe 10 and to display the measurements obtained thereby. The computer 21 may be programmed to calculate and display the area of a lesion or other defect based upon the circumference of width of thereof, as measured by the probe 10.

The probe 10 may be provided with a “start” button 22, which can be pressed to begin recording the x and y coordinates of the probe tip 14 as determined by the image processing device.

The tip of the probe may be detachably mounted on the body of the probe to allow the tip of the probe to be replaced after each surgical use. The tip of the probe, and possibly also the body of the probe, may be formed from a medically approved polymeric material.

The invention is not limited to the embodiment(s) described herein but can be amended or modified without departing from the scope of the present invention. While the above embodiment of the present invention has been described in relation to an arthroscopic probe for measuring the dimensions of cartilage lesions and other defects, it is envisaged that measurement probes in accordance with the present invention may have numerous other applications, for example for the precise measurement of features within an engine manifold, within a pipeline or within any other enclosed or inaccessible space. The materials from which the probe is made and the shape and dimensions of the body and tip of the probe may be modified to suit such different applications. A heavy duty version of the probe may be used for measuring the dimensions of internal features and flaws within pipelines, such as process pipes carrying corrosive chemicals.

1. A probe for measuring features in inaccessible locations comprising:
an elongate body adapted to be inserted into said inaccessible location, a probe tip being defined at a distal end thereof,
a light source for transmitting light from said probe tip to illuminate a surface adjacent the probe tip,
an image sensor for generating an image of the surface adjacent the probe tip and
an image processing device programmed to carry out digital imaging correlation upon subsequent images generated by said optical sensor to determine the motion of the probe tip.

2. A probe as claimed in claim 1, wherein said light source comprises at least one LED or at least one laser diode.

3. A probe as claimed in claim 1, wherein said light source and/or said image sensor are provided within the body of the probe, light being transmitted from the light source to the probe tip and from the probe tip to the optical sensor via respective light guides.

4. A probe as claimed in claim 3, wherein said light guides comprise optical fibres.

5. A probe as claimed in claim 1, wherein said light source and/or said image sensor are provided at or adjacent said probe tip.

6. A probe as claimed in claim 1, wherein said probe tip is provided with a lens for receiving reflected light from said surface and transmitting the resulting image to the image sensor.

7. A probe as claimed in claim 6, wherein said image processing device is programmed to determine when said image is in focus.

8. A probe as claimed in claim 7, wherein an indicator is provided for indicating that said image is in focus, as determined by the image processing device.

9. A probe as claimed in claim 6, wherein the lens is recessed into the tip of the probe such that the distance between the lens and an outermost lip of the tip of the probe is equal to the focal distance of the lens whereby the image of the surface is in focus when the tip of the probe is in contact with the surface.

10. A probe as claimed in claim 1, wherein said image sensor comprises an array of light sensors, each light sensor defining a pixel of the resulting image.

11. A probe as claimed in claim 10, wherein each light sensor comprises an individual photodiode.

12. A probe as claimed in claim 10, wherein said light sensors are arranged in a plurality of rows and columns to define a low resolution image of the surface adjacent the tip of the probe.

13. A probe as claimed in claim 1, wherein said light source is adapted to illuminate said surface at an oblique angle.

14. A probe as claimed in claim 1, wherein the orientation of the probe tip with respect to the probe body can be adjusted by up to 90° to allow the probe to be inserted into said inaccessible location from one side in a direction substantially parallel to the surface.

15. A probe as claimed in claim 14, wherein the probe tip is formed from a flexible or malleable material to facilitate such adjustment.

16. A probe as claimed in claim 1, wherein the tip of the probe is detachably mounted on the body of the probe to allow the tip of the probe to be replaced after each use.

17. A probe as claimed in claim 16, wherein the tip of the probe is formed from a medically approved polymeric material.

18. A probe as claimed in claim 1, wherein the probe comprises an arthroscopic probe for measuring the dimensions of a lesion or other defect in articular cartilage.

19. (canceled)
